(19) United States
(12) Patent Application Publication
(10) Pub. No.: US 2022/0195381 A1
(43) Pub. Date: Jun. 23, 2022

(54) CELL CULTURE SUBSTRATE FOR CULTIVATING ADHERENT CELLS

(71) Applicant: FRAUNHOFER-GESELLSCHAFT ZUR FOERDERUNG DER ANGEWANDTEN FORSCHUNG E.V., Munich (DE)

(72) Inventors: Ruben R. ROSENCRANTZ, Potsdam (DE); Lena THORING, Berlin (DE); Stefan KUBICK, Berlin (DE); Alena Lisa PALKOWITZ, Berlin-Zehlendorf (DE)

(21) Appl. No.: 17/595,429

(22) PCT Filed: May 15, 2020
(86) PCT No.: PCT/EP2020/063637

§ 371 Date: Nov. 16, 2021

(30) Foreign Application Priority Data
May 16, 2019 (DE) 10 2019 112 937.0

(51) Int. Cl. C12N 5/00; C08G 73/02

(52) U.S. Cl. CPC C12N 5/0068; C08G 73/0206; C12N 2539/00; C12N 2533/30; C12N 2533/70; C08G 73/028

(57) ABSTRACT

A cell culture substrate for cultivating adherent cells, including:
a substrate (S),
a polymer (P) comprising amino groups, which is bonded to the substrate, and
a saccharide (Z) having at least two monosaccharide units for attaching the adherent cells,
wherein the saccharide (Z) is covalently bonded to the polymer (P) via the amino groups.

Such a cell culture substrate is suitable for cultivating adherent cells and allows the cells to be detached from the cell culture substrate in a gentle manner by adding a saccharide.

### BACKGROUND AND SUMMARY

The “in vitro” cell cultivation of adherent cells, such as mammal cells, in a laboratory is necessary for pharmaceutical research and for the development of cell therapies. In contrast to a cell suspension, adherent cells require attachment to the surface of a cell culture substrate in order to be able to grow. This cell adhesion is mediated by integrin receptors and transmembrane proteins. If the entire growth surface is covered by cultivated adherent cells, strictly adherent cell lines stop growing. Furthermore, once a closed monolayer of adherent cells has formed in the cell culture vessel, the growth of the cells may slow down and the culture may die. For this reason, the cells are diluted before the maximum density is reached (surface is approximately 70-80% covered), by “passaging” the cells, that is, detaching the cells from the cell culture substrate and bringing the cells into suspension. After appropriate dilution, the cells are introduced into a new cell culture vessel for further cultivation. Thus, for the continued cultivation of the adherent cells, “passaging” is regularly carried out in order to avoid negative consequences for the adherent cells.

A frequently used method of detaching adherent cells from the cell culture substrate is “trypsinization”, in which the serine protease trypsin is used to cleave the surface proteins of the cells and disrupt the adhesion to the cell culture vessel. Since trypsin not only cleaves molecules that are relevant for adherence but also non-specifically cleaves surface proteins of the adherent cells such as growth factors or membrane proteins, which play an essential role in cell metabolism. “Trypsinization” therefore has a negative impact on the metabolism of the adherent cells at least in part, and results in cell stress, and therefore reduces the overall vitality of the adherent cells during “passaging”. An alternative method of detaching adherent cells is to use thermoresponsive polymers which have a switchable hydrophilicity/hydrophobicity as a function of the temperature and bring about the detachment of the cells during “passaging” as a result. The detachment of the cells may, for example, take place as a result of a temperature change from standard culture conditions of 37° C. to 22° C. This change in temperature can also result in changes in the metabolism of the adherent cells and may have an adverse impact on the growth behaviour of the cells.

The object of the present disclosure is to specify a cell culture substrate for cultivating adherent cells, a process for cultivating adherent cells using the cell culture substrate, a use of the cell culture substrate for cultivating adherent cells and a kit for cultivating the adherent cells, which are improved in terms of the disadvantages mentioned above.

Provided is a cell culture substrate for cultivating adherent cells, including:

a substrate (S),

a polymer (P) comprising amino groups, which is bonded to the substrate, and

a saccharide (Z) having at least 2 monosaccharide units for attaching the adherent cells,

wherein the saccharide (Z) is covalently bonded to the polymer (P) via the amino groups.

The advantage of the disclosed cell culture substrate is that as a result of the firm attachment of the saccharide to the polymer comprising amino groups and the bonding of the polymer to the substrate, a good attachment of the adherent cells to the cell culture substrate is achieved. This enables a reliable cultivation of adherent cells. With this cell culture substrate, the cells can be detached from the cell culture substrate easily by adding a saccharide, which competitively disrupts the interactions between the surface proteins of the adherent cells and the cell culture substrate, thereby allowing easy “passaging” of adherent cells. In contrast to conventional processes, the cell metabolism is not impaired since neither the cleaving of the surface proteins of the adherent cells nor a temperature change or a change in the pH of the cell culture medium is required.

At least one monosaccharide unit of the saccharide may be present cyclic as a so-called “hemiacetal”, which facilitates the attachment of the surface proteins of the adherent cells to the saccharide. This monosaccharide unit may be, for instance, a hexose, a saccharide with 6 carbon atoms. In other embodiments, the monosaccharide unite may be a pentose, saccharides with 5 carbon atoms.

According to a further embodiment of the cell culture substrate according to the disclosure, the saccharide includes an open-chain monosaccharide unit, wherein the saccharide is bonded to a secondary or primary amine group of the polymer (P) via this monosaccharide unit. The saccharide of the cell culture substrate according to the disclosure comprises at least two monosaccharide units, since as a rule when the saccharide is linked to the polymer (P) comprising amino groups, one open-chain monosaccharide unit may be linked directly to an amino group of the polymer (P) and this monosaccharide unit is then no longer available, or is only available to a limited extent, for attachment to the adherent cells. Recognition by the adherent cells can then occur via the second monosaccharide unit of the saccharide. This allows an easy attachment of the saccharide to the polymer.

The secondary or primary amine bridge between the open-chain monosaccharide unit of the saccharide and the polymer (P) can, for example, be the product of a reductive amination of an originally reducing monosaccharide unit of the saccharide and an amino group of the polymer. This allows the open-chain monosaccharide unit of the saccharide to attach directly to the amino group of the polymer in an easy manner, without linking linker groups having to be present between the polymer and the saccharide.

The saccharide of the cell culture substrate may, for example, be an oligosaccharide or a polysaccharide, such as an oligosaccharide or polysaccharide having 2 to 500 monosaccharide repeat units, or 2 to 20 monosaccharide repeat units, or 2 to 10 monosaccharide repeat units. In some embodiments, a disaccharide, such as for example lactose, maltose, cellobiose or melibiose may be used. Furthermore, hydrolyzed mannan can also be used. In this case, the saccharide may be a polysaccharide and may have e.g. 50 to 500 mannose repeat units, or 100 to 300 mannose repeat units. The use of polysaccharides such as for example cellulose, starch or amylopectin is also possible.

In a further embodiment of the cell culture substrate according to the disclosure, the substrate comprises or consists of glass or synthetic polymers such as plastic. Glass and synthetic polymers are suitable substrates for the cell culture substrate. The plastics may be, for example, polyolefins, such as polyethylene or polypropylene, or other plastics, such as polystyrene (PS) or polycarbonate (PC), for example. Borosilicate glass, for example, can be considered as the glass.

Glass can be suitable as a substrate for reusable cell culture substrates. For example, these can also be re-sterilized after use (20 min at 121° C.) and then reused.

In some embodiments, in the cell culture substrate according to the disclosure, the substrate is covalently bonded to the polymer (P) comprising amino groups. This allows a reliable attachment of the polymer to the substrate. For instance, the substrate and the polymer may be covalently bonded together in the cell culture substrate via amino groups, amide groups and/or via imine groups. In some aspects, oxygen-containing groups, for example hydroxyl groups, keto groups, carboxylate groups or even radical oxygen groups, may be present on the substrate prior to attachment to the polymer, these groups can be produced on the surface of the substrate for example by means of treatment with an oxygen plasma. These oxygen-containing groups allow an easy attachment to the polymer via the amino groups thereof. In some aspects, the surfaces of polystyrene cell culture substrates may be treated with an oxygen plasma, wherein the aforementioned groups can then be formed. A treatment with oxygen plasma to form functional groups is also possible in the case of glass surfaces.

Furthermore, the polymer of the cell culture substrate may comprise or consist of synthetic polymers comprising amino groups, such as poly(ethyleneimine) (PEI) or poly(amidoamine) (PAMAM). Such polymers comprise a plurality of amino groups and therefore make it easy to bond a plurality of saccharides for attaching the adherent cells. The plurality of amino groups can furthermore also be advantageous for the attachment of the polymer to the substrate.

Chitosan, a polymer comprising polyglucosamine as the amino groups, may also be used. The attachment of the saccharide to the chitosan as the polymer can take place analogously to the polymers already described above by means of reductive amination. Not all of the amino groups of the chitosan are reacted with the saccharide, with the result that amino groups of the chitosan are still available for attachment to the substrate.

Furthermore, branched amino group-containing polymers, so-called dendrimers, can also be used, which on account of their high degree of branching may comprise a large number of amino groups.

In a further embodiment of the cell culture substrate according to the disclosure, the substrate comprises the following general structure of formula I:

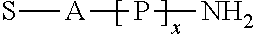

where S is the substrate, P is the polymer with saccharide bonded thereto and A is a group linking the polymer P and the substrate S. The parameter x is a natural integer and represents the number of repeat units in the polymer P. The parameter x may be between 15 and 6,000 repeat units, preferably between 25 and 2,000 repeat units, further preferably between 1,200 and 1,800 repeat units. The PEI used in the embodiment examples comprised, for example, approximately 1,500 repeat units.

Furthermore, the polymer P may comprise repeat units, which are selected from a group of repeat units having the general formulae A to F:

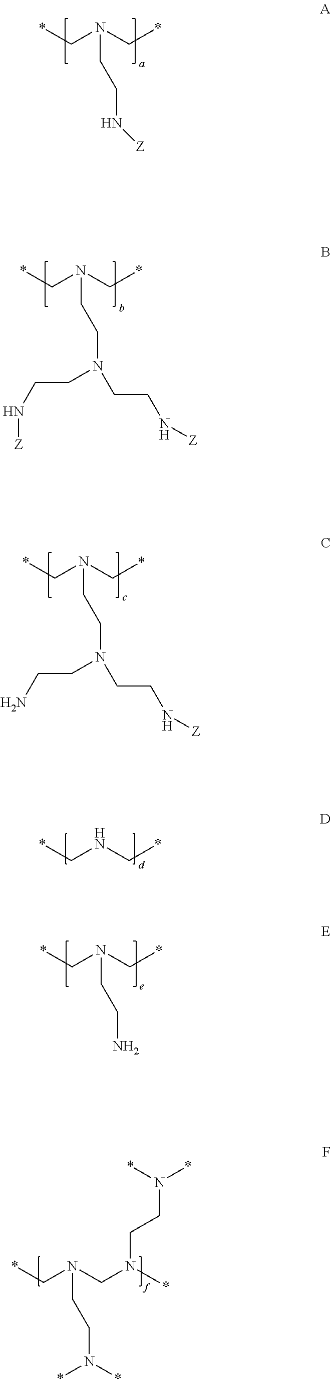

where the group Z represents the saccharide. The parameters a, b, c, d, e and f are, in each case independently of one another, integers and indicate the number of respective repeat units in the polymer P, wherein a+b+c+d+e+f=x. For instance, the parameters a, b, c, d, e and f may, independently of one another, be between 20 and 5,000, or between 25 and 2,000. Often, an average of one saccharide molecule per repeat unit is bonded to the polymer P. In some embodiments, at least the repeat units of the general formulae A, B, and C are present in the polymer and are bonded to one or more saccharides Z. The repeat units of the general formulae D and E represent repeat units in which the amino groups present in the polymer P have not reacted with the saccharide Z. The repeat units F are repeat units with branching points which can be present in the polymer P when branched dendrimers are used as the polymers. The positions marked with “*” indicate the attachment sites of the repeat units to further repeat units in the polymer P, or, in the case of terminal repeat units, the attachment to the group A or to the terminal amino group —NH₂ in the general formula I.

The group A linking the polymer P and the substrate S may be selected from a group consisting of:

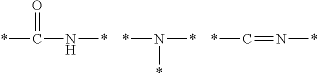

The nitrogen atom of these groups indicates an amino group of the polymer, which reacts with the oxygen-containing groups of the substrate already mentioned above, wherein the corresponding linking groups A are formed. The positions marked with “*” in the groups indicate the attachment sites of the group A to the polymer P and to the substrate S. Such groups may be suitable for forming a stable covalent bond between the polymer P and the substrate S.

The saccharide Z may be selected from a group consisting of the following groups having the formulae G to J:

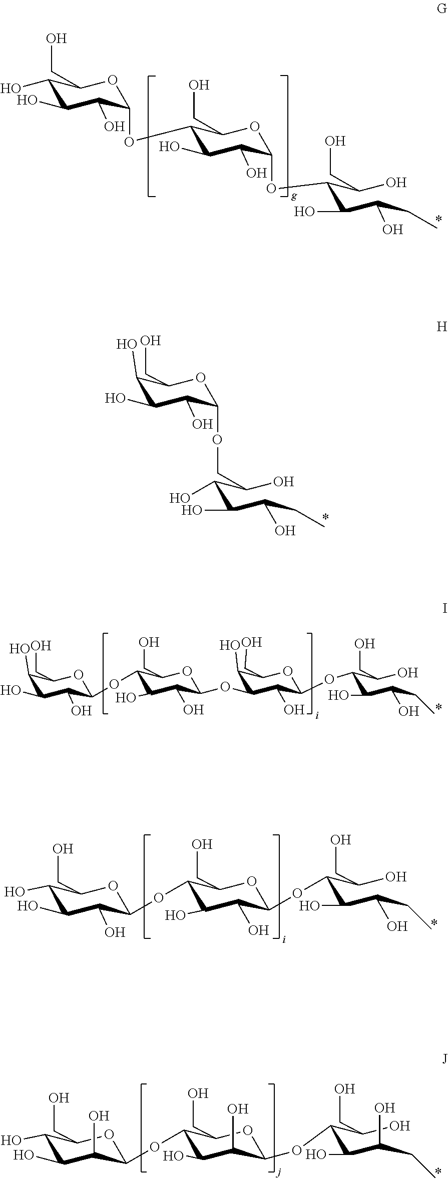

In the formulae, the parameters g, i, and j are, independently of one another, a natural integer between 0 and 400, preferably 0 to 300, further preferably 0 to 10, or are 0 (disaccharide). The positions marked with “*” represent the attachment sites of the saccharide Z to the polymer P.

Generally, the saccharide may comprise glycosidic bonds, such as 1,4- or 1,6-glycosidic bonds, for example β-1,4-glycosidic bonds, α-1,4-glycosidic bonds, and α-1,6-glycosidic bonds. The saccharide Z of the two formulae I comprises, for example, β-1,4-glycosidic bonds, such as those that occur in lactose, or in cellobiose as disaccharides, or in cellulose as polysaccharide. The saccharide of formula G comprises α-1,4-glycosidic bonds, such as those that occur in maltose as disaccharide, for example. Maltose is the breakdown product of the polysaccharide starch by the enzyme beta-amylase. Formula H shows melibiose having an α-1,6-glycosidic bond. Such saccharides Z may be suitable for forming on adherent cells, since many of these structures comprise the monosaccharide galactose, which is recognized by many receptors on the cells. The saccharide of formula J is derived from mannan and may be, for example, the hydrolysis product of the mannan, as described in the embodiment examples.

The cell culture substrate may be formed as a cell culture vessel, for example as a Petri dish or as a microplate. Furthermore, the cell culture substrate may also be formed as particles, for example glass or polystyrene particles on which adherent cells can grow.

A subject-matter of the present disclosure is also a process for producing a cell culture substrate with the process steps of:

providing a polymer (P) comprising amino groups and a saccharide (Z) having at least two monosaccharide units,

forming a covalent conjugate between the polymer (P) and the saccharide (Z),

linking the conjugate to a substrate (S).

For instance, in process step A) a reducing saccharide Z can be provided, and in process step B) the covalent conjugate can be formed by means of reductive amination. Reducing saccharides are monosaccharides, disaccharides, oligosaccharides or polysaccharides which have a free aldehyde group in solution. This aldehyde group reacts with an amino group of the polymer P, wherein the subsequently formed imine group can be reduced to an amine, such as a primary or secondary amine, in mild reducing conditions, for example using sodium cyanoborohydride (NaBH₃CN). Reductive amination is a process that is easy to carry out, in which the polymer comprising amino groups can be covalently bonded directly to the saccharide, without additional linking linker groups having to be present.

In some embodiments, in process step A) a synthetic polymer is provided, which may be poly(ethyleneimine) (PEI) or poly(amidoamine) (PAMAM). The use of branched dendrimers of these polymers is also possible.

In process step C) of a variant of the process according to the disclosure for producing a cell culture substrate, a substrate may be used which has been activated by means of an oxygen plasma and therefore has on the surface functional groups, for example carboxylate groups, keto groups or hydroxide groups, which can react with the amino groups of the polymer P. Such substrates can be suitable for forming covalent bonds between the substrate and the polymer saccharide conjugate via the amino groups of the polymer. To couple the polymer saccharide conjugate to the substrate, the conjugate is incorporated into an aqueous solvent and is brought into contact with the substrate for coupling at elevated temperatures, for example 80° C. For example, the polymer saccharide conjugate is brought into contact with the substrate soon after the activation with the plasma. Further, the polymer saccharide conjugate can be coupled to the substrate within 2 to 5 min of the plasma treatment thereof.

Additionally provided is a process for cultivating adherent cells using a cell culture substrate as described above with the process steps of:

bringing the adherent cells into contact with the cell culture substrate, wherein the cells attach to the cell culture substrate via the saccharide,

cultivating the cells with the cell culture substrate in a cell culture medium,

detaching the cells from the cell culture substrate by bringing a saccharide into contact with the cells.

Bringing the cells into contact with the saccharide can be achieved by exchanging the cell culture medium in which the adherent cells are located for a solution containing the saccharide. The saccharide may, for example, be dissolved in an isotonic saline solution or in a cell-free cell culture medium.

Such a process for cultivating adherent cells allows the cells to be easily detached from the cell culture substrate and “passaged” using a saccharide in process step 3). It is not necessary to treat the cells with enzymes, for example trypsin, or to change the temperature in order to detach the cells from the cell culture substrate in process step 3). The pH of the cell culture medium does not have to be changed either, with the result that the cells are detached in a gentle manner without affecting the cell metabolism or the protein expression in the adherent cells as much as is the case with conventional methods of “passaging”.

In some embodiments, process step 3) is initiated when, in process step 2), the cell culture substrate is largely covered, e.g. approximately 70% to 80% covered, with the adherent cells. This can be determined by means of microscopic methods, for example.

Furthermore, in a variant of the cultivation process according to the disclosure, in process step 3) the same saccharide can be used that is also a component of the cell culture substrate. For example, lactose can be used for detaching adherent cells if lactose has been attached to the polymer P as the saccharide in the cell culture substrate. Analogously, maltose, cellobiose or mannose may also be used if these saccharides are components of the cell culture substrate. These saccharides are inexpensive compounds which allow the adherent cells to be detached from the cell culture substrate in process step 3) in an easy and reliable manner Since the adherent cells attach to the cell culture substrate via the same saccharide that is also used to detach the cells, the detachment of the cells can be achieved reliably.

Alternatively, different saccharides from those that are components of the cell culture substrate can also be used to detach the adherent cells from the cell culture substrate. The saccharide used for the detachment should, however, be capable of detaching the adherent cells from the cell substrate in a competitive manner. In this regard it is recommended that for detaching the adherent cells at least those monosaccharides are used that are components of the oligosaccharides or polysaccharides which are present in the cell substrate. If the oligosaccharides or polysaccharides incorporated in the cell culture substrate contain galactose or mannose units, for example, galactose or glucose can also be used as the monosaccharide for detaching the adherent cells.

In process step 3), the saccharide can be used in a concentration of from 5 mM to 25 mM, preferably 5 mM to 10 mM, for detaching the adherent cells from the cell substrate. For this, the saccharide may be dissolved in these concentrations either in PBS or in a cell-free cell culture medium and the existing cell culture medium of a cell culture is exchanged for this solution, which results in the detachment of the cells from the cell culture vessel. At these concentrations, the detachment of the adherent cells from the cell culture substrate can on the one hand be reliably achieved, while on the other hand osmotic stress for the adherent cells, which only occurs at higher concentrations of approximately 100 to 200 mM, can also be avoided. Once process step 3) has been carried out, process steps 1) to 3) can be repeated cyclically one after the other in order to facilitate the continued cultivation of the adherent cells over an extended period.

Depending on the chemical structure of the saccharide, the cell culture substrate according to the disclosure can also be specific to certain cell lines; for example, lactose as the saccharide component of the cell culture substrate is suitable for cultivating CHO cell lines. HeLa cells can be cultivated well on cell culture substrates according to the disclosure which comprise mannan or maltose as the saccharide component.

A subject-matter of the disclosure is also a use of the cell culture substrate as described above for cultivating adherent cells.

A subject-matter of the disclosure is also a kit for cultivating adherent cells, including:

a cell culture substrate as described above, and

a saccharide for detaching the cells from the cell culture substrate.

The kit may, for example, be offered to potential customers as a commercial product in packaging. The saccharide may already be present in the kit as a solution, such as as a stock solution, in an aqueous solution, for example. For instance, a cell culture vessel or polymer particle is used as the cell culture substrate, to which the polymer saccharide conjugate has already been attached. These cell culture vessels or polymer particles may also already be sterilized for immediate use.

Furthermore, the kit may also comprise instructions for carrying out a process for cultivating adherent cells as described above.

### BRIEF DESCRIPTION OF THE FIGURES

In the following, the disclosure will be described in more detail with reference to figures and embodiment examples. There are shown in:

FIG. 1 depicts an example of a synthesis scheme of a polymer saccharide conjugate by means of a reductive amination using NaBH₃CN.

FIG. 2 provides examples of functional groups that can be formed on a polystyrene surface by means of an oxygen plasma treatment.

FIG. 3 depicts a direct covalent bond between the polymer containing amino groups and polystyrene as the substrate.

FIG. 4 is a bar chart showing the results of a lactate dehydrogenase assay for determining cell integrity in adherent cells which have been detached from cell culture plates either by means of a process according to the disclosure or by means of a trypsin treatment.

FIG. 5 is a graph showing the results of a fluorescence test for determining a caspase as evidence of cell integrity in adherent cells which have been detached from cell culture plates either by means of a process according to the disclosure or by means of a trypsin treatment.

FIG. 6A-C are the results of protein SDS gels (A and C) and western blots (B) for determining the surface protein E-cadherin on the surface of adherent cells which have been detached from cell culture plates either by means of a trypsin treatment or by means of a process according to the disclosure.

FIG. 7 is a graph showing growth curves for adherent cells that have been cultivated on different cell culture plates.

### DETAILED DESCRIPTION

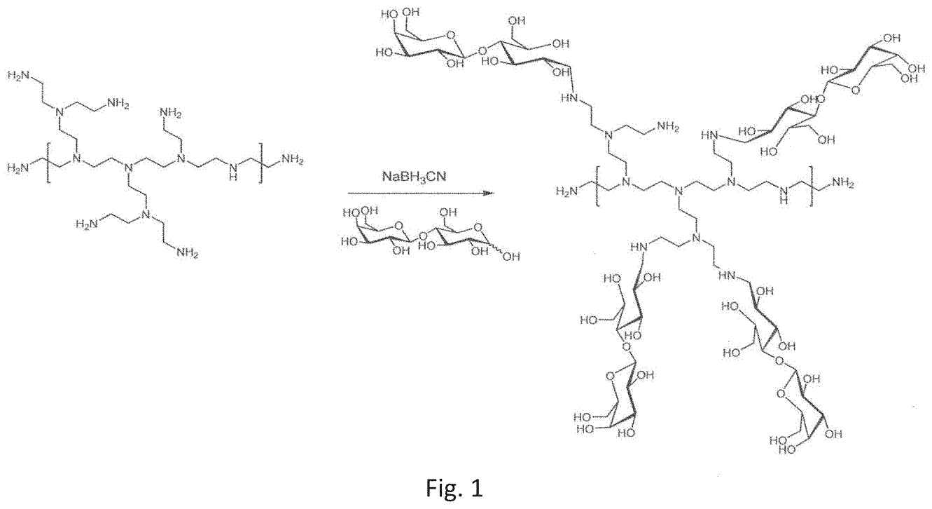

FIG. 1 shows an example of a synthesis of a polymer saccharide conjugate based on the polymer PEI. The saccharide lactose can be linked directly to the amino groups of the PEG by means of reductive amination with NaBH₃CN, wherein one of the cyclic hemiacetal units of the lactose opens and only the galactose remains as a cyclic hemiacetal form and is available for bonding the adherent cells.

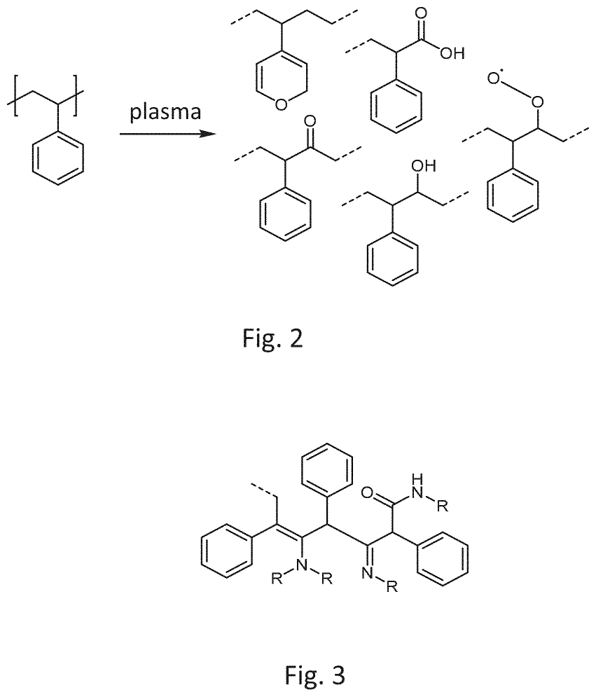

FIG. 2 shows the functional groups formed by means of an oxygen plasma on the surface of polystyrene. For instance, carboxylates, hydroxide groups, and keto groups can be formed. Furthermore, radical oxygen species can also be formed. These groups can react with the amino groups of the polymer and form a permanent covalent bond.

FIG. 3 shows such covalent bonds between an example of a polystyrene substrate and the amino groups of the polymer. For example, imine groups or amine groups can be formed on the amide groups.

### Embodiment Examples

In the following, examples of syntheses of some polymer saccharide conjugates will be presented.

### Lactose PEI:

Polyethylene imine solution (Mn ˜60,000 GPC (gel permeation chromatography), Mw ˜750,000 LS (light scattering spectroscopy), 50 wt. % in water) (PEI, 20 g, 0.1667 mmol) and D-lactose monohydrate (38.028 g, 105.5 mmol, 5 eq. per ideal repeat unit of PEI) are dissolved in MeOH (70 mL) and 50 mM sodium tetraborate solution (aq) (100 mL) and heated to 60° C. Once the components had completely dissolved, the system was cooled down again and the pH was set to a value of 3 with formic acid. NaBH₃CN (33.148 g, 527.5 mmol, 5 eq. based on lactose) was dissolved in MeOH (30 mL) and added to the system in 6 portions. The reaction took place for 2 h at 60° C. Subsequently, the MeOH was removed in vacuo and the solution was dialyzed via a cellulose membrane (exclusion limit 10 kDa) and freeze-dried.

### Maltose PEI:

PEI (10.286 g; see above) and maltose monohydrate (27.124 g, 75.28 mmol; 7.5 eq. per ideal repeat unit of PEI) are completely dissolved in 35 mL MeOH and 5 mM sodium tetraborate solution (aq) at 60° C. After cooling and setting the pH (see above), a suspension of NaBH₃CN (16.5870 g, 263.96 mmol) in 15 mL MeOH was added in portions. The reaction took place for 17 h at 60° C. Subsequently, the MeOH was removed in vacuo and the solution was dialyzed via a cellulose membrane (exclusion limit 10 kDa) and freeze-dried.

### Mannan PEI:

500 mg galactomannan (e.g. locust bean gum or guar gum) is incompletely dissolved in 49 mL H₂O and added to 1 mL 1 M H₂SO₄. The solution is hydrolyzed in the microwave at 600 W for 60 s and then filtered. This results in a hydrolysis of the galactomannan, wherein polysaccharides with 200 to 300 mannose repeat units are formed.

The filtrate is mixed with 1 g PEI, 6 mL glacial acetic acid and 664 mg NaBH₃CN and stirred for 3 h at 60° C. Then, the solution is dialyzed via a cellulose membrane (exclusion limit 10 kDa) and freeze-dried.

### Plasma Treatment of the Substrate and Formation of the Cell Culture Substrate:

The cell culture substrate, for example cell culture plates, can be treated with an oxygen plasma as follows:

Untreated 24-well polystyrene plates are activated with oxygen plasma at 150 W for 90 seconds in vacuo (0.2 mbar) and then 500 μL of a 1 mg/mL aqueous solution of the PEI derivatives (polymer saccharide conjugates) is added to each well. The plates were incubated for 2 h at RT, washed with water and heated, covered, for an hour at 80° C. The plates are then suitable for direct use in the cell culture as cell culture substrates according to the disclosure.

### Cell Culture on Glycopolymer-Coated 24-Well Plates as Cell Culture Substrate According to the Disclosure:

### Media:

CHO-K1 cell lines: Ham's F12 with 10% (v/v) FCS (FCS=foetal calf serum)

HEK293 and HeLa cell lines: DMEM with 10% (v/v) FCS and 2% glutamine

The incubation took place at 37° C. and with a CO₂ content of 5% in the atmosphere. Normally, 0.05-1×10⁶ cells/mL (500 μL) are added to the wells. Passaging is effected by replacing the medium with 500 μL of a 5-50 mM solution of the respective sugar in PBS or medium and incubating for 15-30 minutes at room temperature or 37° C. Depending on the PEI derivative, cell line specificity can be achieved. PEI lactose surfaces are well suited to CHO cell lines, for example. HeLa cells prefer mannan PEI or maltose PEI.

### Tests for Determining Cell Integrity:

In the following, the results of some tests for determining cell integrity after detaching the adherent cells from the cell substrate will be shown. For this, adherent cells which have been detached from a cell substrate according to the disclosure by means of the process according to the disclosure are compared with adherent cells which have been detached by means of a trypsin treatment from conventional cell culture plates that have been surface-modified by means of a plasma. The cells are CHO-1 cells which have been cultivated on polystyrene Petri dishes with PEI lactose.

### Lactate Dehydrogenase Assay for Determining Cell Integrity:

In the event of damage to the plasma membrane, lactate dehydrogenase (LDH) is released from various adherent cells into the cell culture media. The released LDH can be quantified by a coupled enzymatic reaction. First, LDH catalyzes the conversion of lactate into pyruvate by reducing NAD⁺ to NADH. Subsequently, the NADH is used to reduce a tetrazolium salt to a red formazan product by means of the enzyme diaphorase. The quantity of formazan formed, which is determined at a wavelength of 490 nm, is directly proportional to the quantity of released LDH in the medium. The lactate dehydrogenase assay was carried out with a commercially available kit, the “Pierce LDH Cytotoxicity Assay Kit” (Thermo Fisher Scientific, USA).

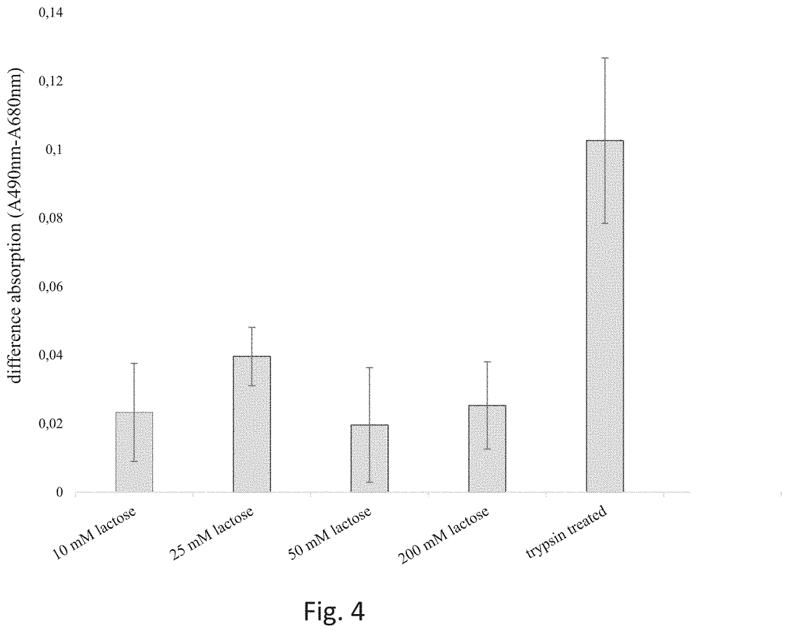

FIG. 4 shows a bar chart in which the difference between the absorption at 490 nm (absorption of formazan) and the absorption at 680 nm (background signal of the instrument) is plotted on the y axis. Listed on the x axis are various ways of detaching adherent cells from the cell substrate using different concentrations of lactose (10 mM, 25 mM, 50 mM and 200 mM) compared with a conventional process, the trypsin treatment.

It is clear to see in FIG. 4 that the lactate dehydrogenase activity in the cell culture medium is much greater in the case of the cells which were treated with trypsin than in the case of the adherent cells which were treated with lactose. This clearly shows that with the present disclosure, cell integrity is less impaired than with the trypsin treatment.

### Caspase Assay for Determining Cell Integrity:

With a fluorescence-based caspase 3/7 assay, apoptosis (programmed cell death) is measured by detecting a caspase in cell cultures. For this, a caspase 3/7 green detection reagent is used, which is a peptide with four amino acids (DEVD) with a cleavage site for caspase 3/7, which is conjugated to a nucleic acid-binding dye. The dye is not fluorescent as long as it is conjugated to the peptide. Once the peptide has been cleaved by the caspase, the dye is activated, binds to DNA and can be detected using fluorescence at an excitation/emission maximum of approx. 502/530 nm.

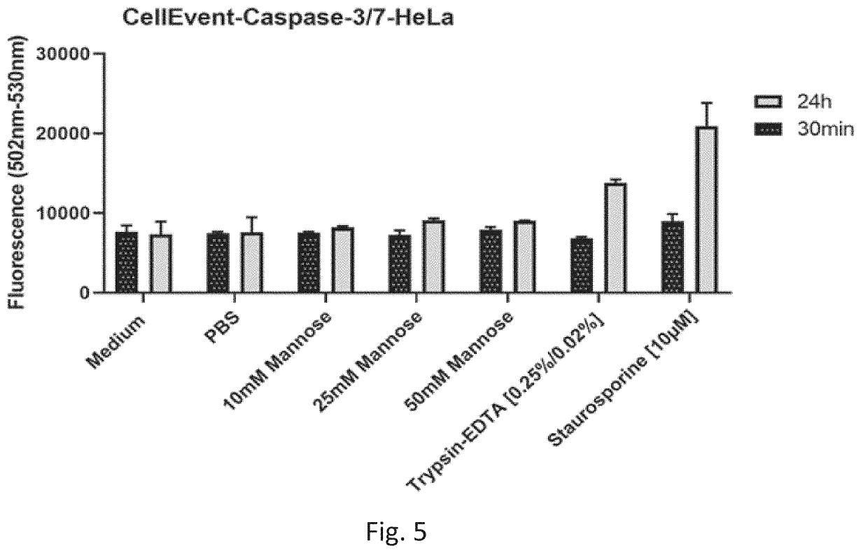

FIG. 5 shows a bar chart in which the fluorescence of the caspase 3/7 green detection reagent is plotted for HeLa cells that have been subjected to different treatments for 30 minutes (black bars) and 24 hours (light bars), respectively. The assay was carried out with the “CellEvent™ Caspase-3/7 Green Detection Reagent” kit (Thermo Fisher Scientific, USA).

In the chart, the bars labelled “Medium” and “PBS” show the caspase-mediated fluorescence of cells that were only washed with the cell culture medium or PBS (phosphate buffered saline), respectively and thus were not exposed to any cell stress at all. The bars labelled “Staurosporine [10 μM]” show the caspase-mediated fluorescence of cells that were treated with staurosporine, a broad-spectrum kinase inhibitor, which triggers apoptosis in many cells. The caspase-mediated fluorescence of HEK cells which were subjected to a trypsin treatment is labelled “Trypsin-EDTA [0.5 and 20%/0.02%]”. Furthermore, the caspase-mediated fluorescence of cells that were cultivated on a cell culture substrate according to the disclosure and then detached by adding a saccharide, namely 10 mM mannose, 25 mM or 50 mM mannose, is shown.

The caspase-mediated activity of the cells not exposed to any cell stress is comparable to the activity of the caspase in adherent cells which were cultivated and detached using a process according to the disclosure. In contrast thereto, the caspase-mediated activity in cells which were subjected to a trypsin treatment is considerably increased. The experimental data of FIG. 5 clearly show that with a cultivation process according to the disclosure, or with the use of a cell culture substrate according to the disclosure, cell integrity is improved compared with a trypsin treatment.

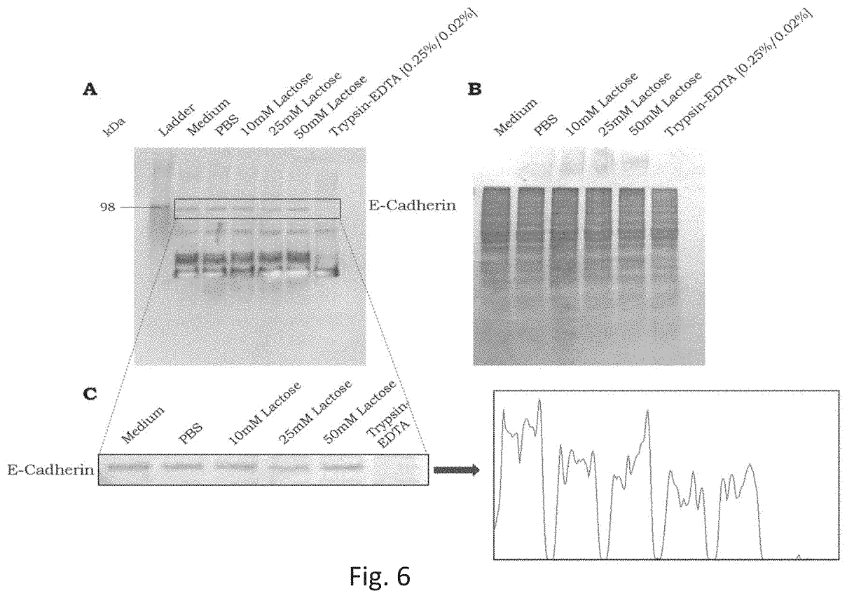

FIG. 6 shows the detection of the protein E-cadherin, a transmembrane glycoprotein, in cells which were exposed to the same conditions as represented in FIG. 5. During the trypsin treatment, E-cadherin was also digested, with the result that this protein is no longer detectable in the SDS protein gel (FIGS. 6A and C). In the case of cells detached using lactose, the band for E-cadherin is still clearly visible, similar to the cells treated with the medium or PBS, which indicates high cell integrity (FIG. 6B). This shows that trypsin digestion also impairs surface proteins that are important for cell metabolism, whereas with the present disclosure this is not the case.

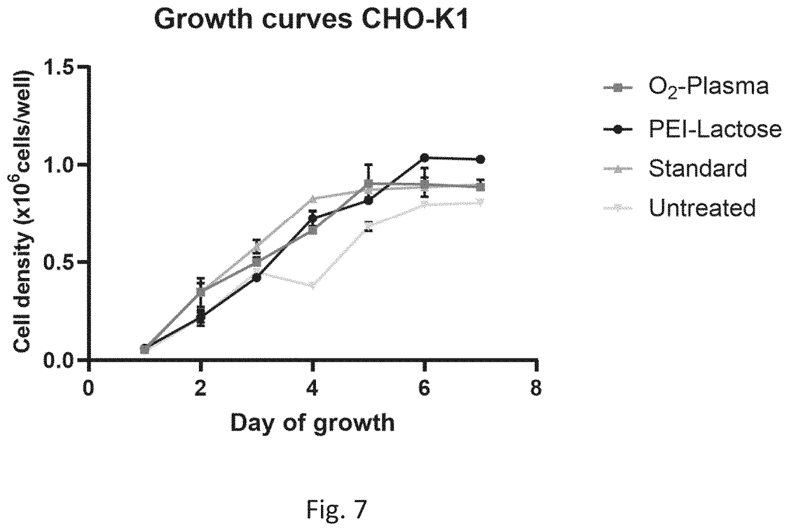

FIG. 7 shows the growth curve of CHO-1 cells on various cell culture vessels. The CHO cells which were cultivated on a cell culture substrate according to the disclosure (polystyrene Petri dishes with PEI lactose) show similar growth curves to cells which were cultivated in conventional cell culture vessels (with growth curves labelled “Standard”). The growth curve labelled “untreated” shows the growth of the cells on untreated cell culture plates which were also used as substrate for the cell culture substrate according to the disclosure in this experiment. The curve labelled “O₂ plasma” shows the growth on cell culture plates which were plasma-treated, but to which the polymer saccharide conjugate was not bonded.

The disclosure is not limited by the description based on the embodiment examples. Rather, the disclosure comprises each new feature and every combination of features, which includes, for instance, every combination of features in the claims, even if this feature or combination is itself not explicitly specified in the claims or embodiment.

1. A cell culture substrate for cultivating adherent cells, comprising
a substrate (S),
a polymer (P) comprising amino groups, which is bonded to the substrate, and
a saccharide (Z) having at least two monosaccharide units for attaching the adherent cells,
wherein the saccharide (Z) is covalently bonded to the polymer (P) via the amino groups.

2. The cell culture substrate according to claim 1, wherein the saccharide comprises an open-chain monosaccharide unit and is bonded to a secondary amine group of the polymer (P) via this monosaccharide unit.

3. The cell culture substrate according to claim 1, wherein the saccharide is an oligosaccharide or polysaccharide having 2 to 500 monosaccharide repeat units.

4. The cell culture substrate according to claim 1, wherein the substrate comprises glass or plastic.

5. The cell culture substrate according to claim 4, wherein the substrate is covalently bonded to the polymer comprising amino groups.

6. The cell culture substrate according to claim 1, wherein the polymer comprises poly(ethyleneimine) (PEI) or poly(amidoamine) (PAMAM).

7. The cell culture substrate according to claim 1, comprising the following structure of the general formula I: 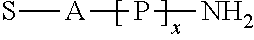
wherein S represents the substrate, P represents the polymer with saccharide bonded thereto and A represents a group linking the polymer P and the substrate S,
wherein x is a number of repeat units in the polymer P,
wherein the polymer P comprises repeat units which are selected from a group of repeat units having the general formulae A to F: 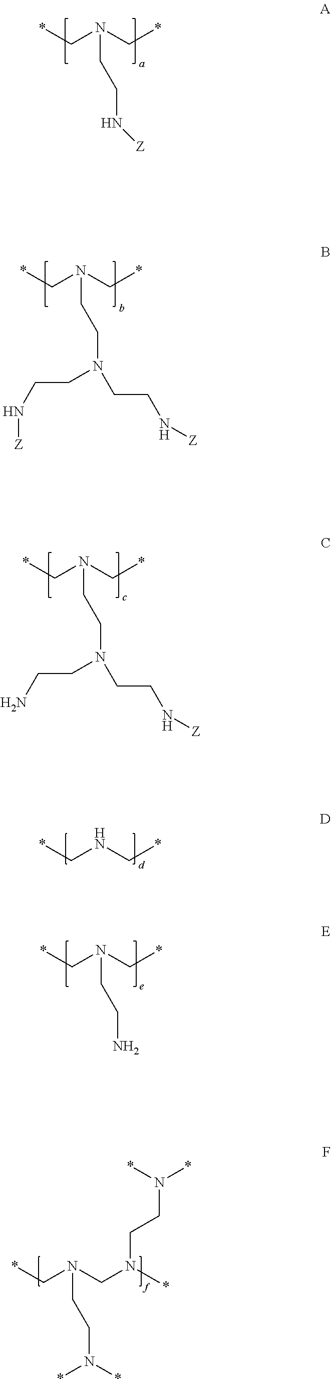
wherein Z represents the saccharide, and a, b, c, d, e and f are a number of respective repeat units in the polymer P, wherein a, b, c, d, and e, are independently selected integers, a+b+c+d+e+f=x, and “*” represents attachment sites of the repeat units to further repeat units in the polymer P or, when the repeat units are terminal repeat units, the attachment to the group A or to a terminal amino group —NH₂ in the general formula I.

8. The cell culture substrate according to claim 7, wherein the group A linking the polymer P and the substrate S is selected from a group consisting of the following groups: 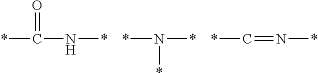
wherein “*” represents the attachment sites of the group A to the polymer P and the substrate S.

9. The cell culture substrate according to claim 1, wherein the saccharide Z is selected from a group consisting of the following groups having the formulae G to J: 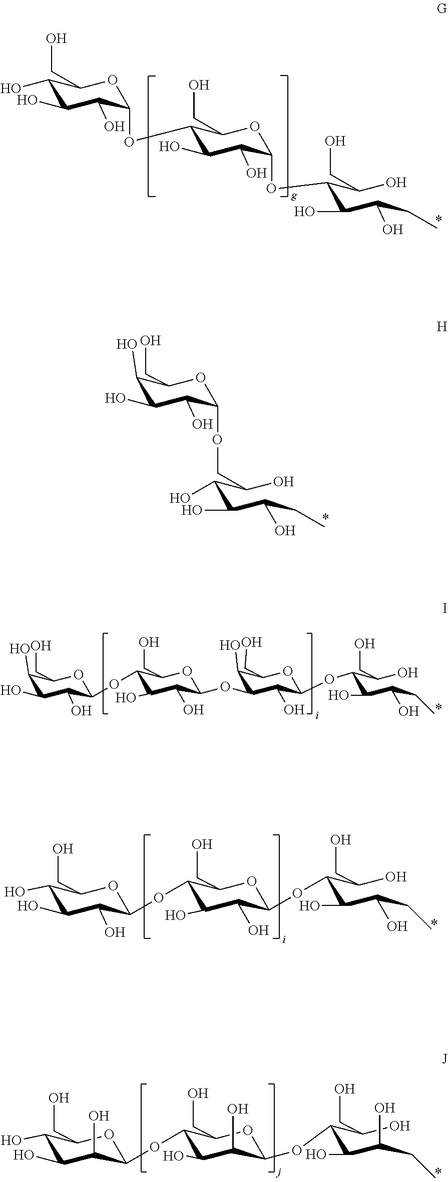
wherein parameters g, i and j are, independently of one another, a natural integer between 0 and 400, and
wherein “*” represents attachment sites of the saccharide Z to the polymer P.

10. The cell culture substrate according to claim 1, formed as a cell culture vessel or as a particle.

11. A process for producing a cell culture substrate comprising:
A) providing a polymer (P) comprising amino groups and a saccharide (Z) having at least two monosaccharide units,
B) forming a covalent conjugate between the polymer (P) and the saccharide (Z), and
C) linking the conjugate to a substrate (S).

12. The process according to claim 11, wherein in step A) the saccharide (Z) is a reducing saccharide, and in step B) the covalent conjugate is formed by means of reductive amination.

13. The process according to claim 11, wherein the polymer (P) in step A) is a synthetic polymer.

14. The process according to claim 11, wherein in step C) the substrate has been activated by means of an oxygen plasma.

15. The process according to claim 14, wherein in step C) covalent bonds are formed between the substrate and the polymer saccharide conjugate via the amino groups of the polymer (P).

16. The process for cultivating adherent cells using the cell culture substrate according to claim 1 comprising:
1) bringing the adherent cells into contact with the cell culture substrate, wherein the cells attach to the cell culture substrate via the saccharide,
2) cultivating the cells with the cell culture substrate in a cell culture medium,
3) detaching the cells from the cell culture substrate by bringing a saccharide into contact with the cells.

17. The process according to claim 16, wherein in process step 3) the saccharide is a component of the cell culture substrate.

18. The process according to claim 16, wherein in process step 3) the saccharide is used in a concentration of from 5 to 25 mM.

19. (canceled)

20. A kit for cultivating adherent cells, comprising:
a cell culture substrate according to claim 1, and
a saccharide for detaching the cells from the cell culture substrate.

21. (canceled)

22. The process according to claim 13, wherein the synthetic polymer is poly(ethyleneimine) (PEI) or poly(amidoamine) (PAMAM), which may also be branched.
